(19) United States
(12) Patent Application Publication
(10) Pub. No.: US 2025/0104828 A1
(43) Pub. Date: Mar. 27, 2025

(54) ESTIMATION OF QUANTITATIVE SYSTEMS PHARMACOLOGY (QSP) TREATMENT EFFECT PARAMETERS USING DEEP LEARNING

(71) Applicant: Genentech, Inc., South San Francisco, CA (US)

(72) Inventor: James LU, San Francisco, CA (US)

(21) Appl. No.: 18/961,292

(22) Filed: Nov. 26, 2024

Related U.S. Application Data
(63) Continuation of application No. PCT/US23/67779, filed on Jun. 1, 2023.
(60) Provisional application No. 63/347,831, filed on Jun. 1, 2022.

(51) Int. Cl. G16H 20/10; G06N 3/0455; G16H 50/20; G16H 50/50

(52) U.S. Cl. CPC G16H 20/10; G06N 3/0455; G16H 50/20; G16H 50/50

(57) ABSTRACT

A method and system for evaluating a treatment using one or more model parameters associated with a quantitative systems pharmacology (QSP) system. Input data corresponding to a treatment is received. The input data is sent to a machine learning system that has been trained, the machine learning system representing at least a portion of the QSP model. The machine learning system is used to generate a set of values for a set of treatment effect parameters associated with the QSP model. A final output is generated based on an evaluation of the treatment on a subject using the set of values for the set of treatment effect parameters.

### CROSS-REFERENCE TO RELATED APPLICATIONS

The present application is a continuation of International Application No. PCT/US2023/067779 filed on Jun. 1, 2023, which claims priority to U.S. Provisional Application No. 63/347,831, filed Jun. 1, 2022, and titled, “ESTIMATION OF QUANTITATIVE SYSTEMS PHARMACOLOGY (QSP) TREATMENT EFFECT PARAMETERS USING DEEP LEARNING,” the entirety of which is incorporated herein by reference.

### FIELD

The present disclosure generally relates to quantitative systems pharmacology (QSP) models and more specifically, to estimating QSP model parameters using deep learning.

### BACKGROUND

Quantitative systems pharmacology (QSP) is a mechanistically oriented form of treatment and disease modeling that is increasingly proving to be impactful in treatment (e.g., drug) discovery and development. For example, QSP modeling can be used in the early stages of drug discovery and/or the later stages of drug development and life-cycle management. QSP models integrate treatment features with target biology and various effectors of interest. Such treatment features may include, for example, but are not limited to, dose, dosing regimen, exposure or concentration at a target site, potency, and other such features. Effectors of interest may include, for example, one or more downstream effectors at the molecular, cellular, and/or pathophysiological levels; one or more functional effectors such as physiologically-based pharmacodynamic study end points (e.g., longitudinal tumor size); or a combination thereof. Accordingly, QSP models may generally be used to generate hypotheses and support a quantitative understanding of one or more novel compound mechanisms of action in a specific tissue, disease, experimental patient population, or clinical patient population context.

### SUMMARY

Methods, systems, and articles of manufacture, including computer program products, are provided for estimating QSP parameters. In one aspect, there is provided a method for evaluating a treatment using one or more model parameters associated with a quantitative systems pharmacology (QSP) model. The method may include receiving input data corresponding to the treatment. The method may also include sending the input data to a trained machine learning model. The trained machine learning model may approximate at least a portion of the QSP model. The method may also include generating, via the trained machine learning model, a set of values for a set of treatment effect parameters associated with the QSP model. The set of values are configured to be used to evaluate the treatment on a subject.

In one aspect, there is provided a system. The system may include at least one processor and at least one memory. The at least one memory may store instructions that result in operations when executed by the at least one processor. The operations may include: receiving input data corresponding to a treatment. The operations may also include sending the input data to a trained machine learning model. The trained machine learning model may approximate at least a portion of a quantitative systems pharmacology (QSP) model. The operations may also include generating, via the trained machine learning model, a set of values for a set of treatment effect parameters associated with the QSP model. The set of values are configured to be used to evaluate the treatment on a subject.

In another aspect, there is provided a computer program product that includes a non-transitory computer readable storage medium. The non-transitory computer-readable storage medium may include program code that causes operations when executed by at least one processor. The operations may include: receiving input data corresponding to a treatment. The operations may also include sending the input data to a trained machine learning model. The trained machine learning model may approximate at least a portion of a quantitative systems pharmacology (QSP) model. The operations may also include generating, via the trained machine learning model, a set of values for a set of treatment effect parameters associated with the QSP model. The set of values are configured to be used to evaluate the treatment on a subject.

In some variations, one or more of the features disclosed herein including the following features can optionally be included in any feasible combination.

In some variations, the trained machine learning model comprises a neural network.

In some variations, the treatment includes a molecule. The molecule is at least one of a micro molecule having a molecular weight of less than 1000 Daltons and a macro molecule having a molecular weight of greater than or equal to 1000 Daltons.

In some variations, the method and/or operations includes predicting an effect of the treatment on the subject using the set of values for the set of treatment effect parameters.

In some variations, the method and/or operations includes generating an adjusted dosing schedule for the treatment based on the evaluation of the treatment on the subject using the set of values for the set of treatment effect parameters.

In some variations, the method and/or operations includes generating an adjusted treatment plan for the treatment based on the evaluation of the treatment on the subject using the set of values for the set of treatment effect parameters.

In some variations, the method and/or operations includes predicting dose response data for the treatment using the trained machine learning model and the set of values for the set of treatment effect parameters.

In some variations, receiving the input data includes receiving dose response data. The dose response data includes a dose response of a plurality of cell types to the treatment.

In some variations, generating, via the trained machine learning model, the set of values for the treatment effect parameters includes generating, via the trained machine learning model, at least one of an Emax value or an EC50 value for each of a plurality of cell types.

In some variations, the method and/or operations includes training the machine learning model. In some variations, the training includes generating simulated dose response data and simulated treatment effect data, training a decoder of the machine learning model using the simulated treatment effect data to generate reconstructed dose response data, fixing a plurality of weights of the decoder that has been trained, and training an encoder of the machine learning model using the decoder having the plurality of weights that have been fixed to generate reconstructed treatment effect data. A difference between the simulated dose response data input to the encoder and the reconstructed dose response data output from the decoder may be minimized.

In one aspect, there is provided a method for evaluating a treatment using one or more model parameters associated with a quantitative systems pharmacology (QSP) model. The method includes generating simulated dose response data and simulated treatment effect data for the treatment. The method may also include training an encoder and a decoder of a machine learning model using the simulated dose response data and the simulated treatment effect data to minimize a difference between the simulated dose response data input to the encoder and reconstructed treatment effect data output from the decoder. The method may also include receiving dose response data corresponding to the treatment. The method may also include sending the dose response data to the trained encoder. The method may also include generating, via the trained encoder, a set of values for a set of treatment effect parameters associated with the QSP model. The method may also include generating a final output based on an evaluation of the treatment on a subject using the set of values for the set of treatment effect parameters.

In one aspect, there is provided a system. The system may include at least one processor and at least one memory. The at least one memory may store instructions that result in operations when executed by the at least one processor. The operations may include: generating simulated dose response data and simulated treatment effect data for the treatment. The operations may also include training an encoder and a decoder of a machine learning model using the simulated dose response data and the simulated treatment effect data to minimize a difference between the simulated dose response data input to the encoder and reconstructed treatment effect data output from the decoder. The operations may also include receiving dose response data corresponding to the treatment. The operations may also include sending the dose response data to the trained encoder. The operations may also include generating, via the trained encoder, a set of values for a set of treatment effect parameters associated with the QSP model. The operations may also include generating a final output based on an evaluation of the treatment on a subject using the set of values for the set of treatment effect parameters.

In another aspect, there is provided a computer program product that includes a non-transitory computer readable storage medium. The non-transitory computer-readable storage medium may include program code that causes operations when executed by at least one processor. The operations may include: generating simulated dose response data and simulated treatment effect data for the treatment. The operations may also include training an encoder and a decoder of a machine learning model using the simulated dose response data and the simulated treatment effect data to minimize a difference between the simulated dose response data input to the encoder and reconstructed treatment effect data output from the decoder. The operations may also include receiving dose response data corresponding to the treatment. The operations may also include sending the dose response data to the trained encoder. The operations may also include generating, via the trained encoder, a set of values for a set of treatment effect parameters associated with the QSP model. The operations may also include generating a final output based on an evaluation of the treatment on a subject using the set of values for the set of treatment effect parameters.

In some variations, one or more of the features disclosed herein including the following features can optionally be included in any feasible combination.

In some variations, the trained encoder includes a neural network system.

In some variations, generating the simulated dose response data and the simulated treatment effect data includes: generating the simulated dose response data and the simulated treatment effect data using a reference model that comprises a plurality of ordinary differential equations (ODEs).

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 is a block diagram of a treatment evaluation system in accordance with various embodiments.

FIG. 2 is a schematic diagram of a process for training a decoder in accordance with various embodiments.

FIG. 3 is a schematic diagram of a process for training an encoder in accordance with various embodiments.

FIG. 4 is an illustrative representation of dose response data in accordance with various embodiments.

FIG. 5 is an illustrative representation of effect data in accordance with various embodiments.

FIG. 6 is a flowchart of a process for evaluating a treatment using one or more model parameters associated with a QSP model in accordance with various embodiments.

FIG. 7 is a flowchart of a process for training a machine learning model in accordance with various embodiments.

FIG. 8 is a flowchart of a process for evaluating a treatment using one or more model parameters associated with a QSP model in accordance with various embodiments.

FIG. 9 is a block diagram of a computer system in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

One of the primary challenges in QSP modeling is estimating the model parameters for the QSP model. Currently available approaches can be time-consuming, inefficient, and be computational resource-expensive. For example, determining QSP model parameters from experimental data may currently perform computationally intensive global optimization operations that include multiple iterations and that take significant time and computing resources.

### I. Overview

Recognizing the increasing importance of QSP modeling in treatment discovery and treatment development, the embodiments described herein provide one or more methods, systems, or both for predicting model parameters for building QSP models. A QSP model may track cell pharmacokinetics within a patient's body after administration of a molecule to the patient to effectively study one or more treatments including the molecule. The QSP model can be described by one or more model parameters and may accurately predict distributions of cell phenotypes in and/or across various physiological compartments over time and/or patient responses, over time.

The system consistent with implementations of the current subject matter may significantly reduce the time and processing resources to estimate model parameters. Thus, the system described herein may be used to accurately and efficiently predict model parameters that, in some instances, can then be used to predict patient responses to potential treatments, ideal patients for clinical trials, predict a change in patient responses over time, and/or the like.

In some instances, not all laboratories or clinical research sites may have access to the type of computer resources needed to perform the computations involved in some of the currently available methodologies for estimating QSP model parameters to study treatment effects (e.g., drug effects). Further, because performing such computations can be time-consuming, clinical trial or clinical research groups may be unable to use the information resulting from these methodologies to make early or on-demand adjustments to treatments, determine how a patient will respond to a treatment, and/or the like. Still further, some of the current methodologies for estimating QSP model parameters may require one or more persons with a certain level of knowledge, skill, or both to make decisions, perform certain actions, or a combination thereof during the process itself.

Thus, the embodiments described herein provide methods and systems for estimating QSP model parameters (e.g., treatment effect parameters, disease parameters, and/or other suitable types of model parameters) to evaluate a treatment (e.g., a drug treatment, a radiation therapy treatment, a chemotherapy treatment, a T-cell therapy, and/or other suitable types of treatments). Implementing the systems described herein to perform model parameter estimation may reduce the computational resources needed to effectively evaluate treatment effects (e.g., drug effects), as well as the amount of time needed to perform this estimation. Using the system described herein may reduce or, in some cases, eliminate the knowledge or skill needed to perform the estimation. Further, using the system described herein to perform model parameter estimation may help solve the computer-related problem of reducing the computational and processing resources used to perform model parameter estimation.

For example, some currently available methodologies for identifying model parameters involve using global optimization algorithms that take input data, perform simulations and evaluations to generate parameters, and then make improvements for the next iteration of input to output. This series of steps may be performed every time that data is provided. In some cases, there may be a variable number of iterations depending on the input data provided. Examples of algorithms used by currently available methodologies include, for example, without limitation, Expectation-Maximization (EM) algorithms, Scatter Search algorithms, and Genetic Algorithms. Thus, as the input data changes, the computational effort required to perform model parameter estimation varies and is often very high.

The embodiments described herein reduce the overall computational and processing resources employed relative to conventional methods. For example, the input data to output parameter mapping may be performed via a single iteration without requiring a new mapping for different input data. The same computational effort is expended for any input. Further, the improved speed and efficiency provided by the embodiments described herein may enable real-time applications.

Thus, the embodiments described herein provide an improved way of automating, using deep learning, QSP model parameter estimation that reduces the overall time and computing resources associated with this estimation. Because the embodiments described herein reduce the amount of computing resources used to estimate QSP model parameters, the methods and systems described herein may be more accessible to and repeatable by various laboratories, clinical research sites, etc. Further, once QSP model parameters have been estimated, a QSP model can be built using these parameters and used to evaluate a treatment, which may, in turn, lead to various actions being taken. For example, using the methods and/or systems described herein may sufficiently speed up the process of evaluating treatment effects so as to enable a treatment protocol (e.g., schedule, dosing, etc.) to be adjusted during the earlier stages of a clinical trial.

One example of a mechanism that can be modeled using QSP modeling is hematopoiesis, the production of the various cellular components of blood and blood plasma. While the example of hematopoiesis is described herein, the system consistent with implementations of the current subject matter can be used for predicting model parameters for other mechanisms, including, T cell therapies, drug therapies, small molecule-based therapies, large molecule-based therapies, disease treatment therapies, among others, that can be modeled. During the example of hematopoiesis, immature precursor cells develop into mature blood cells. The monophyletic theory posits that a single type of stem cell gives rise to all of the mature blood cells in the body. For example, a hematopoietic stem cell may give rise to a multipotential progenitor (MPP) cell from which different types of lineage-committed progenitor cells are created. These lineage-committed progenitor cells may, in turn, give rise to precursor cells, which later become mature blood cells. Mature blood cells include, for example, monocytes, granulocytes, erythrocytes, platelets, natural killer (NK) cells, B lymphocytes, T lymphocytes, and/or other suitable types of cells. Once these mature blood cells have completed their individual life cycles, they become dead cells.

Drugs can affect hematopoiesis in various undesirable ways. Some currently available methods for studying the effects of drugs on hematopoiesis include identifying, for a given point in time, the number of cells of each type at the varying stages of hematopoiesis. Further, certain drug effect parameters may be studied including, for example, E_{MAX} and E_{C50}. E_{MAX} (or Emax) is the maximum effect of a drug. E_{C50} (or EC50) is the concentration of a drug that produces 50% of that drug's maximal effect. Currently available methods for studying the effects of drugs on hematopoiesis can be more computationally intensive, time-consuming, tedious, and expensive with respect to resources consumed than desired. Such issues may negate or at least reduce the usefulness of these available methods.

Thus, the embodiments described herein provide an improved way of evaluating treatment effects (e.g., drug effects) on hematopoiesis using machine learning (e.g., deep learning) to reduce the time and computing resources associated with this evaluation. For example, without limitation, a neural network system may be used to evaluate treatment effects based on dose response data. The dose response data may include a dose response of a plurality of cell types that include the different cell types at the different stages of hematopoiesis for the various different cell lineages. The dose response data is input to the neural network system to generate values for treatment effect parameters, such as Emax and EC50, among others.

The system described herein can estimate QSP model parameters using machine learning such as deep learning. The estimation of QSP model parameters for a QSP model to study drug effects on hematopoiesis, as described herein, may be but one example of how the various embodiments described herein may be implemented. The embodiments described herein may be used to estimate QSP model parameters for other types of QSP models to evaluate, for example, other treatment effects with the same or similar time and resource savings.

### II. Exemplary Terms and Context

The disclosure is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion.

In addition, as the terms “on,” “attached to,” “connected to,” “coupled to,” or similar words are used herein, one element (e.g., a component, a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

The term “subject” may refer to a subject of a clinical trial, a person undergoing treatment, a person undergoing anti-cancer therapies, a person being monitored for remission or recovery, a person undergoing a preventative health analysis (e.g., due to their medical history), or any other person or patient of interest. In various cases, “subject” and “patient” may be used interchangeably herein.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology and toxicology are described herein are those well-known and commonly used in the art.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” can be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, a “model” may include one or more algorithms, one or more mathematical techniques, one or more machine learning algorithms, or a combination thereof.

As used herein, “machine learning” is the practice of using algorithms to parse data, learn from it, and then make a determination or prediction about something in the world. Machine learning uses algorithms that can learn from data without relying on rules-based programming. Deep learning is one type of machine learning.

As used herein, an “artificial neural network” or “neural network” (NN) refers to mathematical algorithms or computational models that mimic an interconnected group of artificial neurons that processes information based on a connectionistic approach to computation. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network processes information in two ways; when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

As used herein, an “encoder” is a type of neural network that learns to efficiently encode data (e.g., input data such as, for example, dose response data) into a vector of parameters (e.g., one or more QSP model parameters such as, for example, treatment effect parameters) having a number of dimensions. The number of dimensions may be preselected.

As used herein, a “decoder” is a type of neural network that learns to efficiently decode a vector of parameters (e.g., one or more QSP model parameters such as, for example, treatment effect parameters) having a number of dimensions (e.g., a number of preselected dimensions) into output data (e.g., dose response data).

As used herein, a “treatment” means a drug or drug treatment, a radiation treatment, a chemotherapy treatment, some other type of treatment, or a combination thereof. A drug treatment may include, for example, the administration of one or more drugs (or therapeutics) simultaneously, at different times, or both.

### III. Exemplary Systems for Treatment Evaluation Using Quantitative Systems Pharmacology (QSP) Modeling

### A. System in General

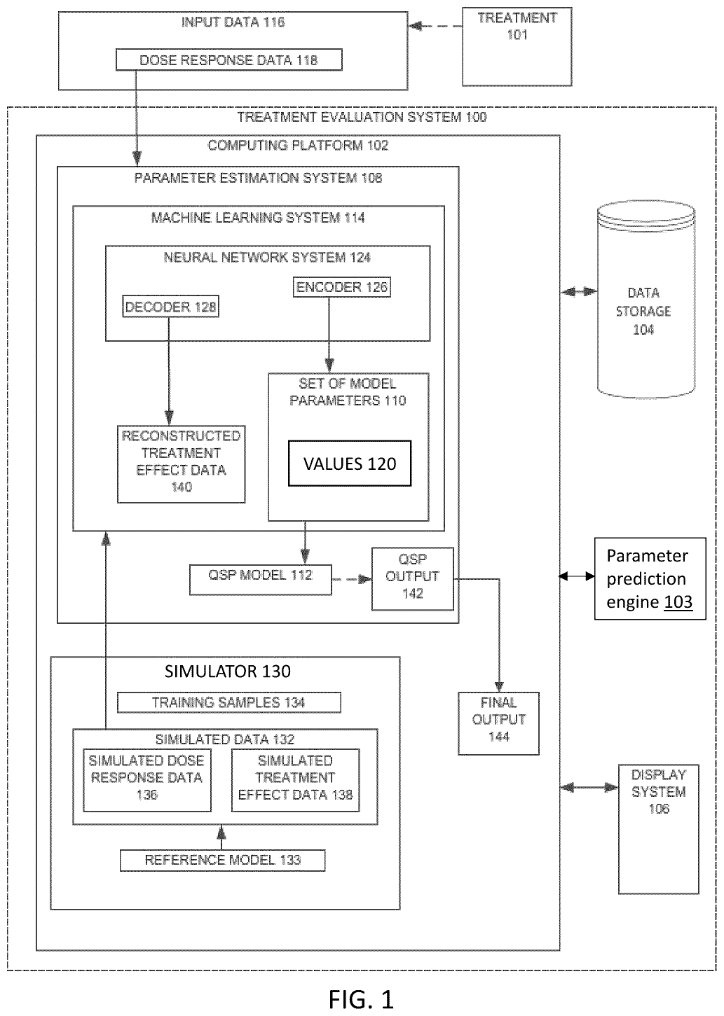

FIG. 1 is a block diagram of a treatment evaluation system 100 in accordance with various embodiments. The treatment evaluation system 100 is used to evaluate the treatment 101. For example, the treatment evaluation system 100 may be used to evaluate the effects of the treatment 101 on the human body. The treatment 101 may include administration of a molecule to a patient. The molecule is at least one of a micro molecule having a molecular weight of less than 1000 Daltons and a macro molecule having a molecular weight of greater than or equal to 1000 Daltons. The molecule may be delivered to a patient to achieve a desired therapeutic effect.

The treatment 101 may include a drug treatment, a radiation treatment, a chemotherapy treatment, a T cell treatment, and/or other suitable types of treatments. In one or more embodiments, the treatment 101 may be any type of or combination of treatments can be administered to a human body.

In one or more embodiments, the treatment evaluation system 100 includes computing platform 102, data storage 104, and display system 106 (see FIG. 1). Computing platform 102 may take various forms. In one or more embodiments, computing platform 102 includes a single computer (or computer system) or multiple computers in communication with each other. In other embodiments, computing platform 102 takes the form of a cloud computing platform. As shown in FIG. 1, computing platform 102 may include a parameter estimation system 108, a machine learning system or machine learning model 114, a neural network system (e.g., a neural network) 124, and/or the like. In some embodiments, the computing platform 102 additionally and/or alternatively includes a simulator 130 (see FIG. 1). The parameter estimation system 108, the machine learning model 114, the neural network system 124, and the simulator 130 may be separately coupled, integrated together, and/or implemented by or as part of a parameter prediction engine 103 (see FIG. 1). The parameter prediction engine 103 includes at least one data processor and at least one memory storing instructions, which when executed by the at least one data processor, perform one or more operations as described herein.

Data storage 104 and display system 106 are each in communication with computing platform 102. In some examples, data storage 104, display system 106, or both may be considered part of or otherwise integrated with computing platform 102. Thus, in some examples, computing platform 102, data storage 104, and display system 106 may be separate components in communication with each other, but in other examples, some combination of these components may be integrated together.

Referring to FIG. 1, treatment evaluation system 100 includes parameter estimation system 108 that is used to estimate set of model parameters 110 associated with Quantitative Systems Pharmacology (QSP) model 112. QSP model 112 is a mechanistically oriented model that integrates treatment features (e.g., after administration of a molecule to a patient) with target biology and various effectors of interest. Such treatment features may include, for example, but are not limited to, dose, dosing regimen, exposure or concentration at a target site, potency, and other such features. Effectors of interest may include, for example, one or more downstream effectors at the molecular, cellular, and/or pathophysiological levels; one or more functional effectors such as physiologically-based pharmacodynamics study end points (e.g., longitudinal tumor size); or a combination thereof.

The set of model parameters 110 may include one or more model parameters, such as one or more treatment effect parameters, one or more disease parameters, and/or other suitable types of model parameters. The set of model parameters 110 for the QSP model 112 may include, for example, treatment, drug effect parameters, disease parameters, and/or other suitable types of model parameters. The one or model parameters can be adjusted to accurately predict the impact of varying dose, cell phenotype ratios (e.g., compositions) of various therapies, and/or the like.

The one or more disease parameters may include systems-specific parameters, patient-specific parameters, and/or other suitable types of disease parameters. Thus, the one or more disease parameters may include values that describe various aspects of a patient, including a patient's demographic information, age, gender, weight, height, history, physiology, and/or other suitable types of disease parameters.

The one or more treatment effect parameters (e.g., dose response data) may include one or more pharmacokinetic parameters, such as a set of cellular kinetics parameters which describe the response of cells of the patient to a molecule. The one or more pharmacokinetic parameters may be used to determine an activity of the molecule in the body of the patient, which can in turn be used to determine the efficacy and/or therapeutic effect of the molecule. The one or more pharmacokinetic parameters may additionally and/or alternatively be used for molecule design.

For example, the treatment effect parameters can include a maximum effect of a drug or agent (Emax), a concentration of the drug or agent that produces 50% of that drug's maximal effect (EC50), an area under curve (also referred to as “AUC”), a maximum plasma concentration (also referred to as “Cₘₐₓ”) of a molecule, a minimum plasma concentration (also referred to as “Cₘᵢₙ”), a half-life (also referred to as “t_{1/2}”) of a molecule, a time of maximum concentration (also referred to as “Tₘₐₓ”) of a molecule, a mean residence time (also referred to as “MRT”), and/or other suitable types of treatment effect parameters.

Emax or E_{MAX} may be the maximum effect of the molecule. E_{C50} or EC50 is the concentration of a molecule that produces 50% of that molecule's maximal effect. The area under curve may be an area under a plasma concentration curve associated with a model (e.g., the QSP model described hrein). The plasma concentration curve may describe plasma concentration in the blood plasma of the patient as a function of time after a dose of a molecule is delivered to the patient. The maximum plasma concentration of the molecule may refer to a maximum (e.g., a peak) plasma concentration in the blood plasma of the patient after delivery of the molecule to the patient. The maximum plasma concentration of the molecule may refer to the maximum plasma concentration after a single dose and/or after multiple doses of the molecule. The minimum plasma concentration of the molecule may refer to a minimum (e.g., a trough) plasma concentration in the blood plasma of the patient after delivery of the molecule to the patient. The minimum plasma concentration of the molecule may refer to the minimum plasma concentration after a single dose and/or after multiple doses of the molecule. The time of maximum concentration of the molecule may refer to a time point associated with the maximum plasma concentration. In other words, the time of maximum concentration may be a time point at which the plasma concentration in the blood plasma of the patient has reached the maximum plasma concentration after a single dose and/or multiple doses of the molecule. The half-life of the molecule may refer to a time for a dose of the molecule in the blood plasma of the patient after delivery of the molecule to the patient to reach one-half of its steady-state value. The mean residence time may refer to an average time the molecule remains in the body of the patient after delivery of a single dose and/or multiple doses of the molecule to the patient.

The treatment effect parameters may additionally and/or alternatively include the behavior of the cell phenotypes after delivery of the molecule to the patient. The cell phenotypes may include a stem cell, a hematopoietic stem cell, a multipotential progenitor cell, lineage-committed progenitor cells, precursor cells, mature blood cells (e.g., monocytes, granulocytes, erythrocytes, platelets, natural killer (NK) cells, B lymphocytes, T lymphocytes, and/or other suitable types of mature blood cells), dead cells, and/or other suitable types of cell phenotypes. The set of cellular kinetics parameters includes a quantity, a proliferation (e.g., expansion) rate, an apoptosis rate, a trafficking rate, a differentiation rate of the cell phenotypes, and/or other suitable types of cellular kinetics parameters.

Parameter estimation system 108 may be implemented using hardware, software, firmware, or a combination thereof, such as by the parameter prediction engine 103. In one or more embodiments, parameter estimation system 108 is implemented in computing platform 102.

Parameter estimation system 108 includes machine learning model 114. In one or more embodiments, machine learning model 114 includes a deep learning model or system that includes any number of deep learning models and/or algorithms. A deep learning system may be implemented using, but is not limited to, one or more neural networks. For example, the deep learning system may include a neural network system comprised of any number of or combination of neural networks. For example, a neural network system may include one or more neural networks, each of which includes or more neural networks itself.

Parameter estimation system 108 receives input data 116 that corresponds to treatment 101 (see FIG. 1). Input data 116 may be generated in various ways. For example, input data 116 may be generated in a clinical setting, a preclinical setting, an in vitro setting, or a combination thereof. As one specific example, input data 116 may be generated from a biological sample (e.g., blood draw sample) taken from a subject undergoing treatment 101. The subject may be a person. This sample may be taken in relation to a clinical trial for a particular drug treatment protocol. This sample may be taken as a baseline sample prior to or at the beginning of the clinical trial, during the clinical trial, or after the clinical trial.

Input data 116 may take various forms relating to one or more of the treatment features described above. For example, input data 116 can include dose response (or drug response) data 118. In one or more embodiments, dose response data 118 (e.g., as shown in FIG. 1) describes a response of a plurality of different cell types (e.g., cell phenotypes) to treatment 101. The different cell types may include, for example, without limitation, cells at different stages of hematopoiesis and of different lineages. For example, an erythrocyte and an erythroblast may be considered two different cell types because they represent different stages of hematopoiesis even though they are of the same lineage (e.g., an erythroblast becomes an erythrocyte). In one or more embodiments, input data 116 is generated using flow cytometry. For example, without limitation, input data 116 may identify a number of cells (or cell count) for each of the different cell types using flow cytometry.

Parameter estimation system 108 generates a set of values 120 for a set of model parameters 110 based on input data 116. Set of model parameters 110 may include one or more parameters relating to or describing the effect of treatment 101. Set of model parameters 110 may include the one or more disease parameters (e.g., one or more systems-specific parameters, patient-specific parameters, etc.) described herein. For example, set of model parameters 110 may include one or more treatment effect parameters. In one or more embodiments, when QSP model 112 is to be used for studying the effects of a drug on hematopoiesis, the one or more treatment effect parameters includes E_{MAX} and EC50 for each of the different cell types. Other examples of parameters are described herein.

In one or more embodiments, machine learning model 114 of parameter estimation system 108 includes neural network system 124. Neural network system 124 may include, for example, at least one machine learning model, at least one neural network, at least one recurrent neural network, and/or other suitable types of machine learning models.

Referring to FIG. 1, neural network system 124 may include, for example, an encoder 126 and a decoder 128 that is used to train encoder 126. In some implementations, the parameter prediction engine 103 trains the encoder 126 and the decoder 128. The parameter prediction engine 103 may train the encoder 126 and the decoder 128 in a sequence. The sequence may include the parameter prediction engine 103 first training the decoder 128 (e.g., as part of a forward model) to generate a representation representing an approximation of the QSP model 112. The approximation of the QSP model 112 includes reconstructed treatment effect data 140 (see FIG. 1). After training the decoder 128, the parameter prediction engine 103 trains the encoder 126 (e.g., as part of an inverse model) to predict one or more model parameters (e.g., the set of model parameters 110), based at least on the reconstructed treatment effect data 140 output by the decoder 128.

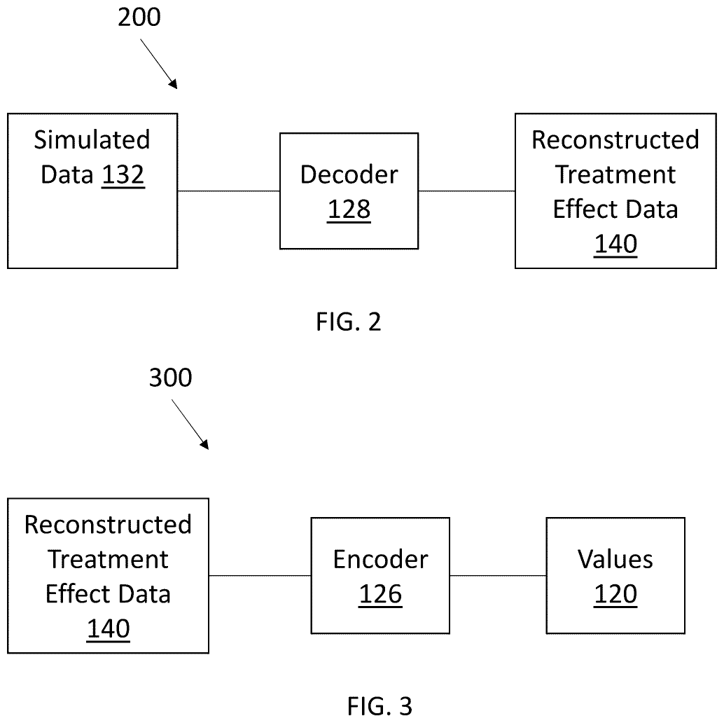

FIG. 2 is a schematic diagram 200 depicting a process for training the decoder 128 as part of the forward model, consistent with implementations of the current subject matter. For example, the parameter prediction engine 103 (shown in FIG. 1) may train the decoder 128 based at least on input data including simulated data 132.

Training of machine learning model 114 (e.g., the decoder 128 shown in FIG. 1) may include using simulator 130 (shown in FIG. 1). Simulator 130, which may be implemented using hardware, software, firmware, or a combination thereof, such as by the parameter prediction engine 103, may be separate from or at least partially integrated within computing platform 102. In one or more embodiments, simulator 130 is considered part of parameter estimation system 108 (shown in FIG. 1). In other embodiments, simulator 130 is considered separate from parameter estimation system 108.

Simulator 130 is used to generate simulated data 132 for use in training machine learning model 114 (e.g., the decoder 128 shown in FIG. 1). In one or more embodiments, simulator 130 uses reference model 133 (shown in FIG. 1) to generate simulated data 132. Reference model 133 may be, for example, without limitation, a non-AI QSP model. In one or more embodiments, reference model 133 is a non-AI QSP model that comprises a plurality of ordinary differential equations (ODEs). Simulated data 132 may be generated for a plurality of training samples 134 (shown in FIG. 1). Training samples 134 may include, for example, hundreds of samples, thousands of samples, tens of thousands of samples, hundreds of thousands of samples, one million samples, tens of millions of samples, etc. The samples may include known ranges for each of the parameters of the non-AI QSP model.

In one or more embodiments, simulated data 132 (shown in FIG. 1) includes, but is not limited to, simulated dose response data 136 and simulated treatment effect data 138 for each training sample of training samples 134, as shown in FIG. 1. Simulated dose response data 136 may be data that is simulated to resemble dose response data 118 (shown in FIG. 1). Where dose response data 118 may include, for example, data snapshots for a certain number of points in time (e.g., 4, 5, 8, 15, 20, etc. snapshots) for a particular treatment protocol, particular person, etc., simulated dose response data 136 may include simulated treatment effect data for hundreds, thousands, or even millions of samples. Simulated treatment effect data 138 may be data that is simulated to resemble set of values 120 (shown in FIG. 1) for one or more treatment effect parameters described above with respect to FIG. 1. Similar to simulated dose response data 136, simulated treatment effect data 138 provides a larger source of training data.

In one or more embodiments, the training of machine learning model 114 includes first training decoder 128 of neural network system 124 using simulated data 132, as shown in FIG. 2. The simulated data 132 may include simulated dose response data 136 and/or simulated treatment effect data 138. The parameter prediction engine 103 may train the decoder 128 to generate a feature representation including one or more features extracted from the simulated data 132. The feature representation may include the reconstructed treatment effect data 140.

In some implementations, the parameter prediction engine 103 fixes the weights and/or parameters of the decoder 128 once the decoder 128 is trained. In some implementations, the parameter prediction engine 103 then trains the encoder 126 (see FIG. 3). Additionally and/or alternatively, the parameter prediction engine 103 trains both encoder 126 and decoder 128 (with decoder 128 having fixed elements) using simulated dose response data 136 as the input for encoder 126 to generate reconstructed dose response data 140 as the output of decoder 128. In this way, the decoder 128 may be used in conjunction with encoder 404 to train, validate, and/or evaluate the performance of the encoder 126 and/or to improve performance of the machine learning model 114. In one or more embodiments, decoder 128 may generate reconstructed dose response data 140 that is within selected tolerances (e.g., within <2%, <3%, <4%, <5%, <6%, <7%, <8%, <9%, or <10%) of the input data that was sent to encoder 126.

This minimizes a difference between simulated dose response data 136 (that is input to encoder 126) and reconstructed dose response data 140 (that is output from decoder 128).

FIG. 3 is a schematic diagram 300 depicting a process for training the encoder 126 as part of the inverse model, consistent with implementations of the current subject matter. For example, the parameter prediction engine 103 (shown in FIG. 1) may train the encoder 126 based at least on input data including the feature representation (e.g., reconstructed treatment effect data 140) generated by the decoder 128. The encoder 126 may be used to transform input data 116 (e.g., dose response data 118 in FIG. 1) into values for a set of QSP model parameters (e.g., set of values 120 for one or more treatment effect parameters). In other words, the parameter prediction engine 103 may train the encoder (e.g., as part of the machine learning model or system 114) to generate one or more values for a set of QSP model parameters that can be used to make predictions about the effects of a molecule after administering the molecule to a patient.

Once trained, neural network system 124 (e.g., the machine learning model) and, in particular, encoder 126 of neural network system 124, may be used to generate set of values 120 for one or more treatment effect parameters, one or more disease parameters, and/or other suitable types of treatment effect parameters, based on dose response data 118 with an improved level of accuracy, speed, and efficiency. Further, a same or similar computational effort may be expended for any given dose response data 118 that is input to neural network system 124. In other words, once the neural network system 124 is trained, the treatment evaluation system 100 may predict values 120 for a set of model parameters 110 based on input data 116, without multiple iterations and with decreased computational resources.

In one or more embodiments, set of values 120 for set of model parameters 110 (e.g., one or more treatment effect parameters, one or more disease parameters, and/or other suitable types of model parameters) may be used to build and/or update QSP model 112 (see FIG. 1). QSP model 112 having set of values 120 for set of model parameters 110 may be used to generate QSP output 142, which includes information that can be important to treatment discovery and development. In one or more embodiments, QSP model 112 evaluates an effect of treatment 101 on a subject to generate treatment features (e.g., dose response data 118) based on set of model parameters 110 (e.g., one or more treatment effect parameters, one or more disease parameters, and/or other suitable types of model parameters). Thus, QSP output 142 may take various different forms.

Parameter estimation system 108 may use set of values 120 generated by machine learning model 114, QSP output 142, or both to identify one or more actions to be performed, generate a final output 144, as shown in FIG. 1, identifying one or more actions to be performed, perform one or more actions based on the information, or a combination thereof. For example, parameter estimation system 108 may use set of values 120 for set of model parameters 110 and/or output 142 to generate a final output (e.g., report) 144 that identifies a change to made to a dosing regimen for treatment 101, a change to made to a treatment schedule for treatment 101, a change in the dosage amount for treatment 101, a change to treatment 101 itself, some other type of information, or a combination thereof. Final output 144 may be displayed on display system 106, stored in data storage 104, sent to and/or displayed on a remote device (e.g., mobile device, tablet, server, laptop, cloud computing device, etc.) over one or more communications links (e.g., wired and/or wireless communications links), or a combination thereof.

### B. Exemplary Configuration for System in Hematopoiesis Context

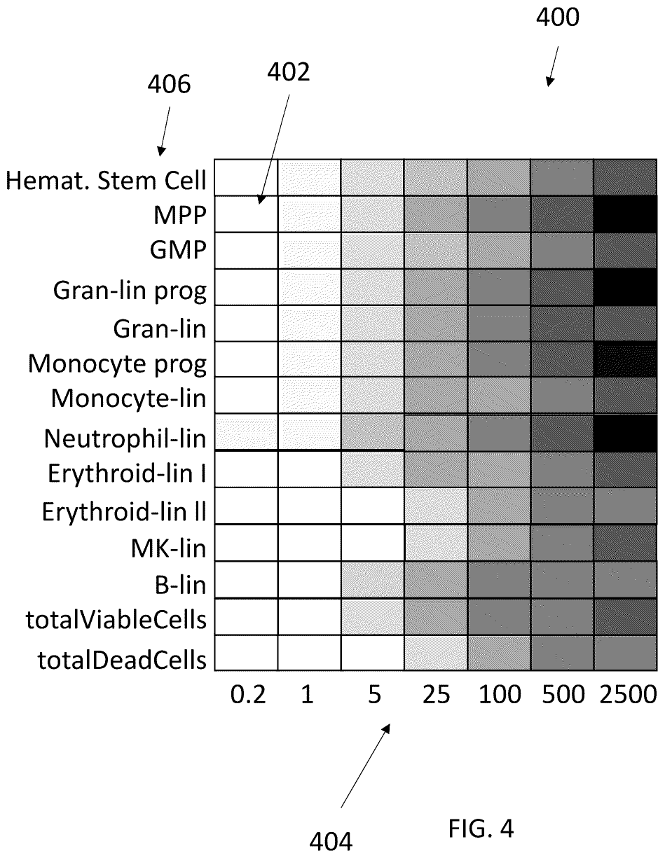

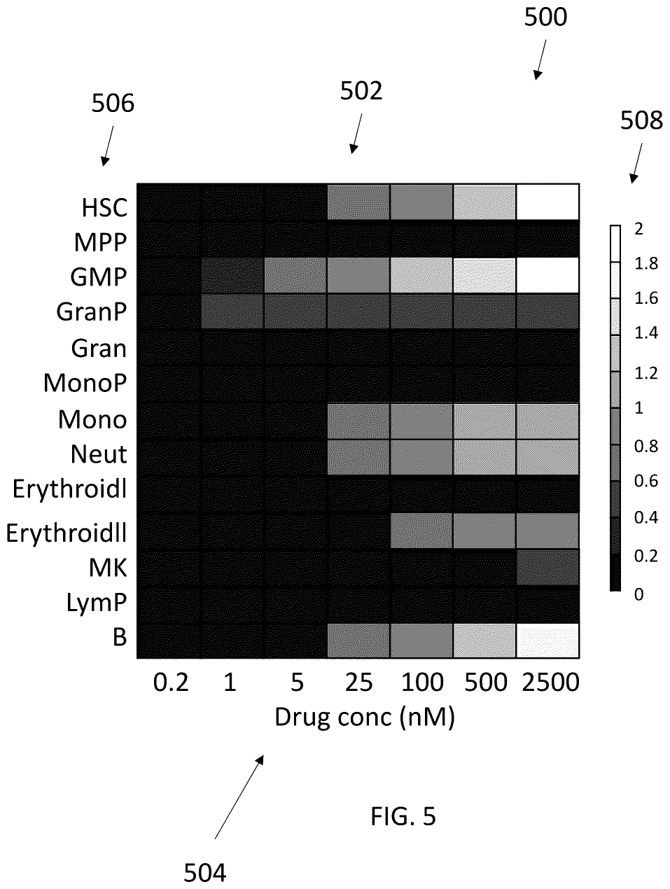

FIGS. 4-5 are illustrations relating to estimating treatment effect parameters with respect to hematopoiesis in accordance with one or more embodiments. These figures are exemplary and are not meant to impose any limitations on the embodiments described herein.

FIG. 4 is an illustrative representation of dose response data 400 in accordance with various embodiments. Dose response data 400 is one example of an implementation for dose response data 118 described with respect to FIG. 1. Dose response data 400 is represented using gradient chart 402 that includes x-axis 404 and y-axis 406. X-axis 404 identifies cell count. The cell count may be determined using flow cytometry. For example, cell count data may be generated via flow cytometry. In one or more embodiments, the cell count day generated via flow cytometry may be normalized or modified in some other manner for representation with respect to x-axis 404.

Y-axis 406 identifies cell type. Each of the different cell types shown in FIG. 4 corresponds to a stage in the life cycle for blood cells. In some examples, two or more cell types of a same cell linage. In some examples, a cell type represents a certain type of cell for a particular cell lineage at a particular stage of hematopoiesis. In other examples, a cell type may be all viable cells, all dead cells, or some other type.

FIG. 5 is an illustrative representation of effect data 500 in accordance with various embodiments. Effect data 500 is one example of an implementation for values 120 for a parameter in set of model parameters 110 described with respect to FIG. 1. Effect data 500 is represented using gradient chart 502 that includes x-axis 504 and y-axis 506. In FIG. 5, x-axis 504 identifies drug concentration; y-axis 506 identifies cell type. Y-axis 506 may correspond with y-axis 506 in FIG. 4. Gradient chart 502 uses the gradients as identified by gradient legend 508 to identify values for the treatment effect parameter, Emax, for the different cell types at the different drug concentrations.

### IV. Exemplary Methodologies for Treatment Evaluation Using Quantitative Systems Pharmacology (QSP) Modeling

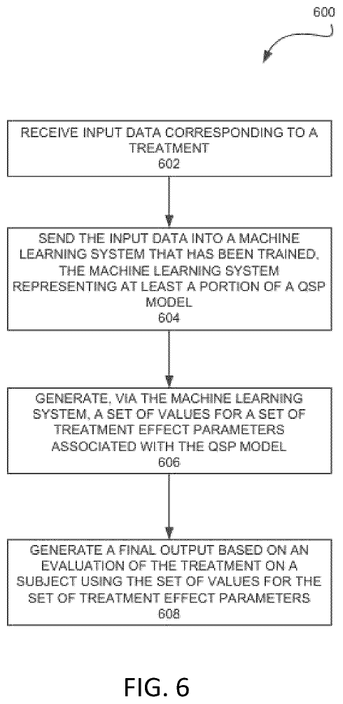

FIGS. 6-8 depict flowcharts for processes 600, 700, 800. Any of the steps of the processes 600, 700, 800 can be interchanged with one another and/or be used in conjunction with one another.

FIG. 6 is a flowchart of a process 600 for evaluating a treatment using one or more model parameters (e.g., the set of model parameters 110 in FIG. 1) associated with a quantitative systems pharmacology (QSP) model in accordance with various embodiments. In various embodiments, process 600 is implemented using treatment evaluation system 100 (e.g., the parameter prediction engine 103 in FIG. 1) described herein. Process 600 may be used to generate respective values for one or more model parameters (e.g., set of model parameters 110 in FIG. 1) associated with the QSP model 112.

Step 602 includes receiving input data corresponding to a treatment. The input data which may take the form of, for example, input data 116 in FIG. 1, includes data related to one or more treatment features. For example, the input data may include dose response data. The dose response data may include a dose response of a plurality of cell types to the treatment. As an example, when studying the effects on hematopoiesis, this type of dose response data may be useful in understanding any hematopoietic effects of a treatment (e.g., drug).

Step 604 includes sending the input data to a machine learning model (e.g., machine learning model 114 in FIG. 1) that has been trained. At least a portion of the machine learning model, such as the decoder 128, represents at least a portion of a QSP model (e.g., the QSP model 112 in FIG. 1). In one or more embodiments, the machine learning model includes a deep learning system. The deep learning system may include, for example, a neural network such as neural network system 124 in FIG. 1.

Step 606 includes generating, via the machine learning model 114, a set of values for a set of model parameters (e.g., set of model parameters 110 in FIG. 1), including one or more treatment effect parameters, one or more disease parameters, and/or other suitable types of model parameters, associated with the QSP model . . . In some embodiments, the set of treatment effect parameters includes at least one of an E_{MAX} value or an EC50 value for each of a plurality of cell types. The set of treatment effect parameters includes one or more treatment effect parameters described herein.

Step 608 includes generating a final output based on an evaluation of the treatment on a subject using the set of values for the set of treatment effect parameters. In some embodiments, the final output (e.g., final output 144 in FIG. 1) includes a report or other visualization. The final output 144 may include, for example, at least one of a prediction about an effect of the treatment on the subject, an adjusted dosing schedule for the treatment, an adjusted treatment plan for the treatment, or some other type of output corresponding to the treatment. For example, step 608 may include predicting an effect of the treatment on the subject using the set of values for the set of treatment effect parameters, generating an adjusted dosing schedule for the treatment based on the evaluation of the treatment on the subject using the set of values for the set of treatment effect parameters, generating an adjusted treatment plan for the treatment based on the evaluation of the treatment on the subject using the set of values for the set of treatment effect parameters, or a combination thereof.

Using process 600 to evaluate the treatment on a subject as described above may reduce the overall time and computing resources that would otherwise be needed to perform such an evaluation using existing methodologies. Process 600 improves the functioning of a computer system by improving its ability to efficiently and accurately estimate a set of values for a set of model parameters (e.g., one or more treatment effect parameters, one or more disease parameters, and/or other suitable types of model parameters) and to generate a final output (e.g., final output 144 in FIG. 1) based on the set of values. For example, via process 600, the input data to output parameter mapping for the QSP model may be performed via a single iteration without requiring a new mapping for different input data. The same computational effort is expended for any input. Because the embodiments described herein reduce the amount of computing resources used to estimate QSP model parameters (e.g., the set of treatment effect parameters), process 600 may be more accessible to and repeatable by various laboratories, clinical research sites, etc.

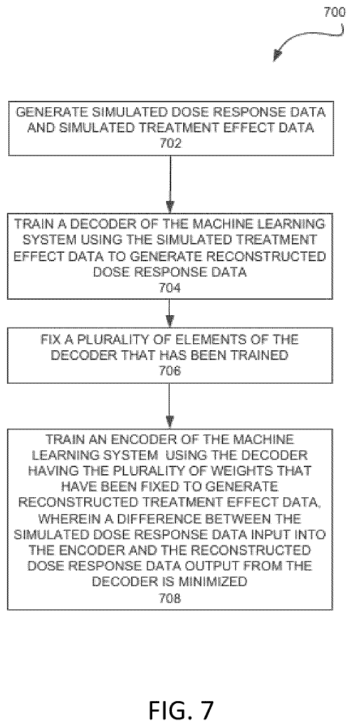

FIG. 7 is a flowchart of a process 700 for training a machine learning model (e.g., the machine learning model 114 in FIG. 1) in accordance with various embodiments. Process 700 describes one example of a manner in which the machine learning model 114 described in process 600 may be trained, such as by the parameter prediction engine 103 shown in FIG. 1. In various embodiments, process 700 is implemented using treatment evaluation system 100 (e.g., the parameter prediction engine 103) described with respect to FIG. 1. This training may include, for example, various deep learning techniques. This training may be performed using, for example, thousands, tens of thousands, hundreds of thousands, or millions of samples of simulated data.

Step 702 includes generating simulated dose response data and simulated treatment effect data. The simulated dose response data (e.g., the simulated dose response data 136 of FIG. 1) and simulated treatment effect data (e.g., the simulated treatment effect data 138 of FIG. 1) may include data for each training sample of a plurality of training samples (e.g., thousands, tens of thousands, hundreds of thousands, or millions of training samples). The simulated dose response data and the simulated treatment effect data may be generated using a reference model (e.g., the reference model 133 of FIG. 1) that includes a plurality of ordinary differential equations (ODEs) or other equations.

Step 704 includes training a decoder of the machine learning model (e.g., the decoder 128 of machine learning model 114 in FIG. 1) using the simulated treatment effect data to generate reconstructed dose response data. In particular, the simulated treatment effect data is input to the decoder of the machine learning model 114. The decoder may include a neural network system. Training the decoder includes minimizing a difference (e.g., an error) between the reconstructed dose response data and the simulated dose response data.

Step 706 includes fixing a plurality of elements of the decoder that has been trained. The elements may be, for example, the weights of the decoder.

Step 708 includes training an encoder of the machine learning model 114 (e.g., the encoder 126 of machine learning system 114 in FIG. 1) using the decoder having the plurality of weights that have been fixed to generate reconstructed treatment effect data, wherein a difference between the simulated dose response data input to the encoder and the reconstructed dose response data output from the decoder is minimized. In step 808, the simulated dose response data is input to the encoder and the encoder outputs the reconstructed treatment effect data (e.g., the reconstructed treatment effect data 140 of FIG. 1). The decoder uses the reconstructed treatment effect data as input to generate the reconstructed dose response data.

After the encoder has been trained, at least the encoder portion of the machine learning model 114 may be used in process 600 as described above with respect to FIG. 6. After training, the trained encoder may be reliably and confidently used to quickly and accurately estimate treatment effect data based on dose response data. Further, using a trained encoder trained via the process 700 described in FIG. 7 may not only improve the efficiency and accuracy of estimating treatment effect data based on dose response data, but may also reduce the overall time and computational resources (e.g., memory, processing power, etc.) that would otherwise be needed to estimating treatment effect data based on dose response data.

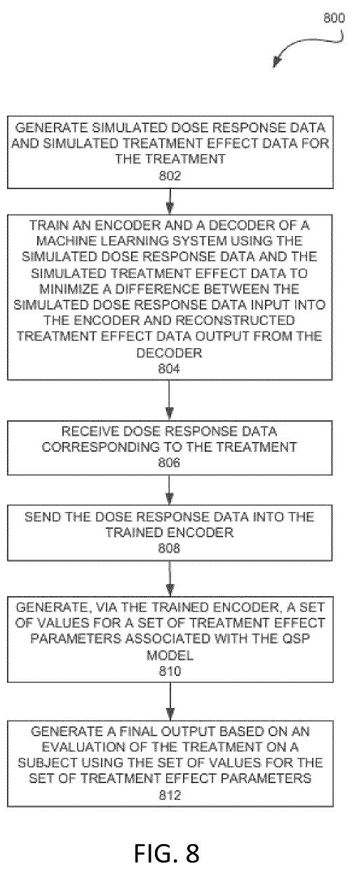

FIG. 8 is a flowchart of a process 800 for evaluating a treatment using one or more model parameters (e.g., the set of model parameters 110) associated with a quantitative systems pharmacology (QSP) model in accordance with various embodiments. In various embodiments, process 800 is implemented using treatment evaluation system 100 (e.g., the parameter prediction engine 103) described herein. Further, process 800 is one example of an implementation for process 600 in FIG. 6 in the context of studying the effects of a treatment (e.g., drug) on hematopoiesis.

Step 802 includes generating simulated dose response data (e.g., the simulated dose response data 136 described with respect to FIG. 1) and simulated treatment effect data (e.g., the simulated treatment effect data 138 of FIG. 1) for the treatment. The simulated dose response data and the simulated treatment effect data may be generated using a reference model (e.g., reference model 133 of FIG. 1) that includes a plurality of ordinary differential equations (ODEs). The simulated dose response data may include dose responses for a plurality of cell types. For example, the simulated dose response data may include cell counts for a plurality of different cell types for a given point in time (e.g., a particular year, a particular month, a particular, day, a particular hour of the day, etc.). The simulated treatment effect data may include, for example, at least one of an E_{MAX} value or an EC50 value for each of the plurality of cell types. The different cell types may include, for example, viable cells, dead cells, granulocytes, erythrocytes, erythroblasts, monocyte progenitor cells, monocytes, etc. Thus, the different cell types may include cell types corresponding to a same or different cell lineage and a same or different stage of hematopoiesis. In one or more embodiments, step 802 may be one example of an implementation for step 702 in FIG. 7.

Step 804 includes training an encoder and a decoder of a machine learning model (e.g., the machine learning model 114) using the simulated dose response data and the simulated treatment effect data to minimize a difference between the simulated dose response data input to the encoder and reconstructed treatment effect data (e.g., the reconstructed treatment effect data 140 of FIG. 1) output from the decoder. In one or more embodiments, step 704 may be one example of an implementation for steps 704, 706, and 708 in FIG. 7.

Step 806 includes receiving dose response data corresponding to the treatment. The dose response data, which resembles the simulated dose response data, may be actual dose response data collected for a subject at a given point in time.

Step 808 includes sending the dose response data to the trained encoder. The dose response data may be, for example, dose response data 118 in FIG. 1.

Step 810 includes generating, via the trained encoder, a set of values for a set of model parameters (e.g., one or more treatment effect parameters, one or more disease parameters, and/or other suitable types of model parameters) associated with the QSP model (e.g., the QSP model 112 of FIG. 1). The set of values for the set of model parameters resembles the simulated treatment effect data. The set of model parameters, such as one or more treatment effect parameters, includes, for example, at least one of an Emax value or an EC50 value for each of the plurality of cell types

Step 812 includes generating a final output based on an evaluation of the treatment on a subject using the set of values for the set of treatment effect parameters. In some embodiments, the final output (e.g., final output 144 in FIG. 1) takes the form of a report. The final output may include, for example, at least one of a prediction about an effect of the treatment on the subject, an adjusted dosing schedule for the treatment, an adjusted treatment plan for the treatment, or some other type of output corresponding to the treatment.

Using process 800, including the trained encoder (e.g., encoder 126 in FIG. 1 that has been trained), to generate set of values for the set of treatment effect parameters and generating the final output (e.g., final output 144 in FIG. 1) based on the set of values for the set of treatment effect parameters may reduce the time and processing (computing) resources that would otherwise be needed to perform such operations using existing methodologies. For example, via process 800, the input data to output parameter mapping may be performed via a single iteration without requiring a new mapping for different input data. The same computational effort is expended for any input. Because the embodiments described herein reduce the amount of computing resources used to estimate QSP model parameters (e.g., the set of treatment effect parameters), process 800 may be more accessible to and repeatable by various laboratories, clinical research sites, etc.

### V. Computer Implemented System

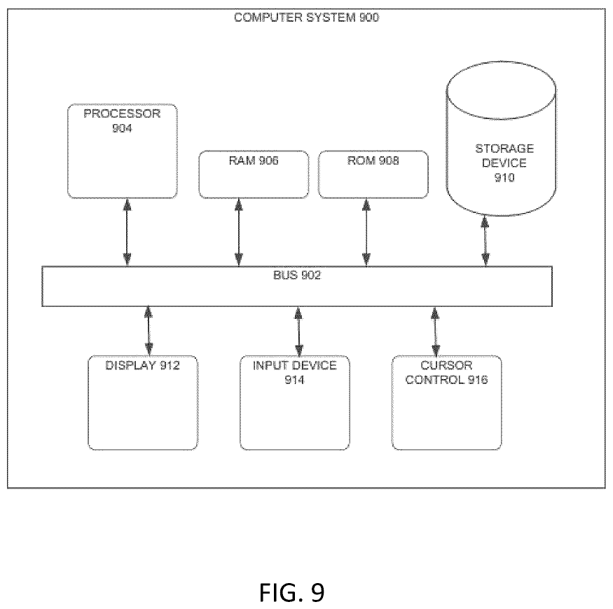

FIG. 9 is a block diagram of a computer system in accordance with various embodiments. Computer system 900 may be an example of one implementation for computing platform 102 (e.g., the parameter prediction engine 103) described herein. In one or more examples, computer system 900 can include a bus 902 or other communication mechanism for communicating information, and a processor 904 coupled with bus 902 for processing information. In various embodiments, computer system 900 can also include a memory, which can be a random access memory (RAM) 906 or other dynamic storage device, coupled to bus 902 for determining instructions to be executed by processor 904. Memory also can be used for storing temporary variables or other intermediate information during execution of instructions to be executed by processor 904. In various embodiments, computer system 900 can further include a read only memory (ROM) 908 or other static storage device coupled to bus 902 for storing static information and instructions for processor 904. A storage device 910, such as a magnetic disk or optical disk, can be provided and coupled to bus 902 for storing information and instructions.

In various embodiments, computer system 900 can be coupled via bus 902 to a display 912, such as a cathode ray tube (CRT) or liquid crystal display (LCD), for displaying information to a computer user. An input device 914, including alphanumeric and other keys, can be coupled to bus 902 for communicating information and command selections to processor 904. Another type of user input device is a cursor control 916, such as a mouse, a joystick, a trackball, a gesture input device, a gaze-based input device, or cursor direction keys for communicating direction information and command selections to processor 904 and for controlling cursor movement on display 912. This cursor control 916 may have two degrees of freedom in two axes, a first axis (e.g., x) and a second axis (e.g., y), that allows the device to specify positions in a plane. However, it should be understood that input devices 914 allowing for three-dimensional (e.g., x, y and z) cursor movement are also contemplated herein.

Consistent with certain implementations of the present teachings, results can be provided by computer system 900 in response to processor 904 executing one or more sequences of one or more instructions contained in RAM 906. Such instructions can be read into RAM 906 from another computer-readable medium or computer-readable storage medium, such as storage device 910. Execution of the sequences of instructions contained in RAM 906 can cause processor 904 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 904 for execution. Such a medium can take many forms, including but not limited to, non-volatile media, volatile media, and transmission media. Examples of non-volatile media can include, but are not limited to, optical, solid state, magnetic disks, such as storage device 510. Examples of volatile media can include, but are not limited to, RAM 906 (e.g., dynamic RAM (DRAM) and/or static RAM (SRAM)). Examples of transmission media can include, but are not limited to, coaxial cables, copper wire, and fiber optics, including the wires that comprise bus 902.

Additionally, a computer-readable medium may take various forms such as, for example, but not limited to, a floppy disk, a flexible disk, hard disk, magnetic tape, or any other magnetic medium, a CD-ROM, any other optical medium, punch cards, paper tape, any other physical medium with patterns of holes, RAM, PROM, EPROM, EEPROM, FLASH-EPROM, solid-state memory, one or more storage arrays (e.g., flash arrays connected over a storage area network), network attached storage, any other memory chip or cartridge, or any other tangible medium from which a computer can read.

In addition to computer readable medium, instructions or data can be provided as signals on transmission media included in a communications apparatus or system to provide sequences of one or more instructions to processor 904 of computer system 900 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 900 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 900, whereby processor 904 would execute the analyses and determinations provided by these engines, subject to instructions provided by any one of, or a combination of, the memory components RAM 906, ROM, 908, or storage device 910 and user input provided via input device 914.

### VI. Additional Considerations

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications, alternatives, and equivalents are possible within the scope of the invention claimed. Thus, it should be understood that although the present invention as claimed has been specifically disclosed by embodiments and optional features, modifications, variations, and/or equivalents of the concepts herein disclosed may be resorted to by those skilled in the art, and that such modifications, variations, and/or equivalents are considered to be within the scope of this invention as defined by the appended claims.

The description provides preferred exemplary embodiments only, and is not intended to limit the scope, applicability, or configuration of the disclosure. Rather, the description of the preferred exemplary embodiments will provide those skilled in the art with an enabling description for implementing various embodiments. It is understood that various changes may be made in the function and arrangement of elements without departing from the spirit and scope as set forth in the appended claims. For example, in describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

Specific details may be provided to provide a thorough understanding of the embodiments. However, it will be understood that the embodiments may be practiced without these specific details. For example, circuits, systems, networks, processes, or other components may be shown as components in block diagram form in order not to obscure the embodiments in unnecessary detail. In other instances, well-known circuits, processes, algorithms, structures, or techniques may be shown without unnecessary detail in order to avoid obscuring the embodiments.

1. A method for evaluating a treatment using one or more model parameters associated with a quantitative systems pharmacology (QSP) model, the method comprising:
receiving input data corresponding to a treatment;
sending the input data into a trained machine learning model, the trained machine learning model approximating at least a portion of the QSP model; and
generating, via the trained machine learning model, a set of values for a set of treatment effect parameters associated with the QSP model, wherein the set of values are configured to be used to evaluate the treatment on a subject.

2. The method of claim 1, wherein the trained machine learning model comprises a neural network.

3. The method of claim 1, wherein the treatment includes a molecule, wherein the molecule is at least one of a micro molecule having a molecular weight of less than 1000 Daltons and a macro molecule having a molecular weight of greater than or equal to 1000 Daltons.

4. The method of claim 1, further comprising:
predicting an effect of the treatment on the subject using the set of values for the set of treatment effect parameters.

5-7. (canceled)

8. The method of claim 1, wherein receiving the input data comprises:
receiving dose response data, wherein the dose response data includes a dose response of a plurality of cell types to the treatment.

9. The method of claim 1, wherein generating, via the trained machine learning model, the set of values for the treatment effect parameters comprises:
generating, via the trained machine learning model, at least one of an Emax value or an EC50 value for each of a plurality of cell types.

10. The method of claim 1, further comprising:
training the machine learning model.

11. The method of claim 10, wherein the training comprises:
generating simulated dose response data and simulated treatment effect data;
training a decoder of the machine learning model using the simulated treatment effect data to generate reconstructed dose response data;
fixing a plurality of weights of the decoder that has been trained; and
training an encoder of the machine learning model using the decoder having the plurality of weights that have been fixed to generate reconstructed treatment effect data, wherein a difference between the simulated dose response data input to the encoder and the reconstructed dose response data output from the decoder is minimized.

12. A system comprising:
at least one data processor; and
at least one memory storing instructions, which when executed by the at least one data processor, result in operations comprising:
receiving input data corresponding to a treatment;
sending the input data into a trained machine learning model, the trained machine learning model approximating at least a portion of a quantitative systems pharmacology (QSP) model; and
generating, via the trained machine learning model, a set of values for a set of treatment effect parameters associated with the QSP model, wherein the set of values are configured to be used to evaluate the treatment on a subject.

13. The system of claim 12, wherein the trained machine learning model comprises a neural network.

14. The system of claim 12, wherein the treatment includes a molecule, wherein the molecule is at least one of a micro molecule having a molecular weight of less than 1000 Daltons and a macro molecule having a molecular weight of greater than or equal to 1000 Daltons.

15. The system of claim 12, wherein the operations further comprise:
predicting an effect of the treatment on the subject using the set of values for the set of treatment effect parameters.

16-18. (canceled)

19. The system of claim 12, wherein receiving the input data comprises:
receiving dose response data, wherein the dose response data includes a dose response of a plurality of cell types to the treatment.

20. The system of claim 12, wherein generating, via the trained machine learning model, the set of values for the treatment effect parameters comprises:
generating, via the trained machine learning model, at least one of an Emax value or an EC50 value for each of a plurality of cell types.

21. The system of claim 12, wherein the operations further comprise:
training the machine learning model.

22. The system of 21, wherein the training comprises:
generating simulated dose response data and simulated treatment effect data;
training a decoder of the machine learning model using the simulated treatment effect data to generate reconstructed dose response data;
fixing a plurality of weights of the decoder that has been trained; and
training an encoder of the machine learning model using the decoder having the plurality of weights that have been fixed to generate reconstructed treatment effect data, wherein a difference between the simulated dose response data input to the encoder and the reconstructed dose response data output from the decoder is minimized.

23. (canceled)

24. A method for evaluating a treatment using one or more model parameters associated with a quantitative systems pharmacology (QSP) model, the method comprising:
generating simulated dose response data and simulated treatment effect data for the treatment;
training an encoder and a decoder of a machine learning model using the simulated dose response data and the simulated treatment effect data to minimize a difference between the simulated dose response data input to the encoder and reconstructed treatment effect data output from the decoder;
receiving dose response data corresponding to the treatment;
sending the dose response data to the trained encoder;
generating, via the trained encoder, a set of values for a set of treatment effect parameters associated with the QSP model; and
generating a final output based on an evaluation of the treatment on a subject using the set of values for the set of treatment effect parameters.

25. The method of claim 24, wherein trained encoder comprises a neural network system.

26. The method of claim 24, wherein generating the simulated dose response data and the simulated treatment effect data comprises:
generating the simulated dose response data and the simulated treatment effect data using a reference model that comprises a plurality of ordinary differential equations (ODEs).

27-30. (canceled)
